(19) United States
(12) Plant Patent Application Publication
(10) Pub. No.: US 2002/0144322 P1
(43) Pub. Date: Oct. 3, 2002

(54) Blackberry plant named 'Zorro'

(76) Inventor: Carlos D. Fear, Aptos, CA (US)

Correspondence Address: PENNIE AND EDMONDS, 1155 AVENUE OF THE AMERICAS, NEW YORK, NY, 100362711

(21) Appl. No.: 09/772,327

(22) Filed: Jan. 29, 2001

(51) Int. Cl. A01H 5/00

(52) U.S. Cl. PLT/203

(57) ABSTRACT

The present invention relates to a new and distinct cultivar of blackberry plant named ‘Zorro’. The new cultivar is distinguished from other blackberry cultivars by its high productivity, early season, and low chill requirement. ‘Zorro’ produces fruit with improved quality and shipping characteristics over a long fruiting period. The new cultivar is distinguished from its seed parent by having better flavored fruit; it is distinguished from its pollen parent by its larger fruit.

### 1. BACKGROUND OF THE INVENTION

This invention relates to a new cultivar of blackberry called ‘Zorro’. The new cultivar was developed from hybridization of the unpatented female cultivar ‘By59.2’ with the unpatented male selection ‘By46.1’. The parents were crossed in Spring 1994 whereafter fruit and seed were collected to produce seedlings for field planting in Watsonville, Calif. in 1994. The new cultivar was selected in 1996 for its good flavor and early season of ripening. The cultivar has been asexually propagated, and reproduced true to type plants by in vitro shoot tip culture.

### 2. SUMMARY OF THE INVENTION

The present invention provides a new and distinct blackberry cultivar named ‘Zorro’. The variety is botanically identified as Rubus L. subgenus Rubus. The new cultivar produces a floricane crop which begins in mid-May and continues until mid-August. The new blackberry variety is distinguished from other varieties by a number of characteristics as set forth in Table 1. In particular, the new cultivar is distinguished by its early season, its low chill requirement, and its improved quality and shipping characteristics. Yield of the new cultivar is high when compared to many other varieties.

### 3. COMPARISON TO SIMILAR VARIETIES

The variety that we believe to be similar to ‘Zorro’ from those known to us is ‘Olaillie’, an unpatented variety. ‘Zorro’ is particularly different from ‘Olaillie’ by being slightly later ripening, having less postharvest color reversion, having less acidic flavor, and having better fruit firmness. Further detailed comparison of ‘Zorro’ to ‘Olaillie’ and ‘Chester’ is presented in Table 1.

### 4. BRIEF DESCRIPTION OF THE DRAWINGS

The accompanying photographs show typical specimens of the fruit, leaves and shoot of the new cultivar, in color as nearly true as reasonably possible in color illustrations of this type.

FIG. 1 is a photograph showing a primocane shoot, mature leaf and stem of ‘Zorro’.

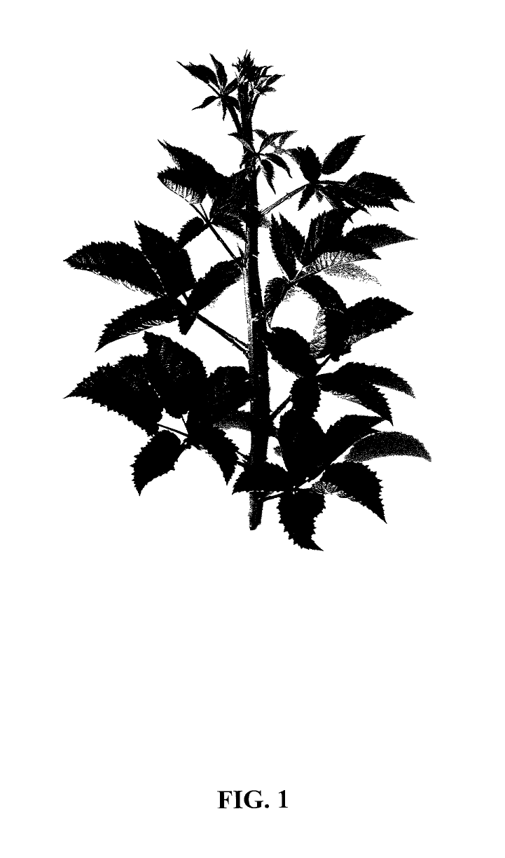

FIG. 2 is a photograph showing a close-up view of a primocane shoot, mature leaf and stem of ‘Zorro’.

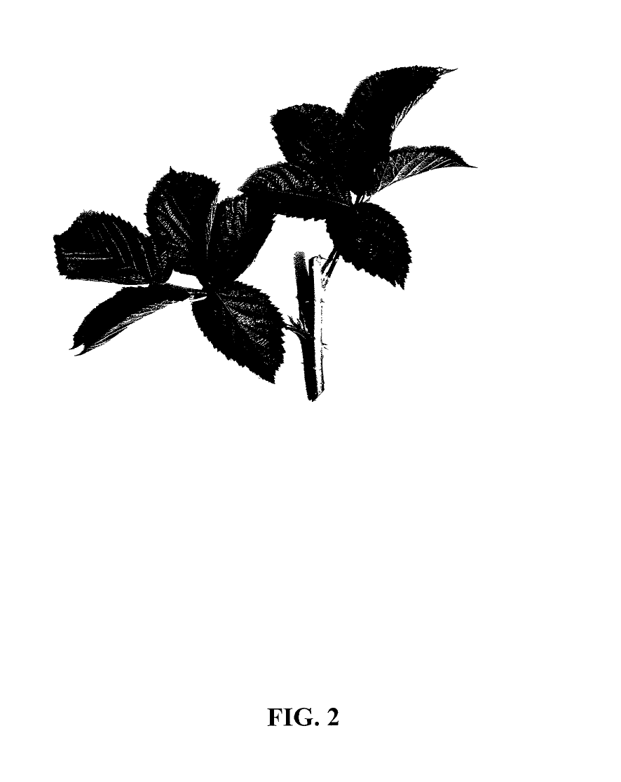

FIG. 3 is a photograph of a ‘Zorro’ fruiting lateral with fruit in various stages of development.

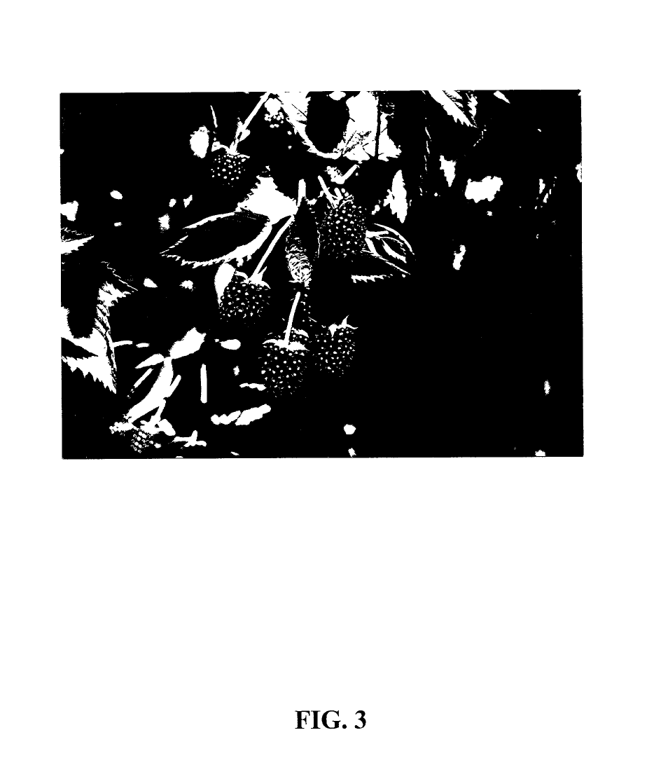

### 5. DESCRIPTION OF THE NEW VARIETY

The following detailed description of the new blackberry cultivar, ‘Zorro’, is based upon recorded observations of plants and fruit grown between 1996 and 2000 in Watsonville, Calif., and is believed to apply to plants of the ‘Zorro’ cultivar grown in similar conditions of soil and climate elsewhere. This description is in accordance with terminology used by the International Union for the Protection of New Varieties of Plants (UPOV). Throughout this specification, color names beginning with a small letter signify that the name of the color, as used in common speech, is aptly descriptive. Color data beginning with a capital letter and followed by an alphanumeric code indicate the most similar color designations as provided by The Royal Horticultural Society (RHS) Colour Chart published by The Royal Horticultural Society of London, England. Color designations, color descriptions, and other phenotypical descriptions may deviate from the stated values and descriptions depending upon variation in environmental, seasonal, climatic and cultural conditions.

### 5.2 CHARACTERISTICS OF THE NEW VARIETY

Table 1 provides information on the plant and fruit characteristics of the new blackberry cultivar ‘Zorro’ compared with characteristics of the unpatented blackberry cultivars ‘Olallie’ and ‘Chester’. Both ‘Olallie’ and ‘Chester’ are currently important cultivars for fresh market shipping, and thus are comparable to the proposed use of the new invention, ‘Zorro’. Observations of ‘Zorro’ and ‘Olallie’ were taken in side-by-side comparison in 1999 and in 2000.

Fruit of the new cultivar is particularly characterized and distinguished from other cultivars by its improved flavor and shipping characteristics. Drupelets of ‘Zorro’ fruit show slightly less postharvest color reversion compared to ‘Olallie’, the most comparable cultivar fruiting at the same time of the season.

‘Zorro’ is highly productive and produces most of its crop in the early part of the harvest season. Canes of ‘Zorro’ are vigorous, thorny and have buds with a low chill requirement.

‘Zorro’ is distinguished from its pollen parent, By46.1, by having larger fruit. ‘Zorro’ is distinguished from its seed parent, ‘By59.2’, by being thorny and having better flavored fruit.

TABLE 1
PLANT CHARACTERISTICS OF ‘ZORRO’

| | Zorro | Olallie | Chester |
|---|---|---|---|
| GENERAL | | | |
| Vigor | moderate-high | moderate-high | high |
| Growth habit | semi-upright | trailing | semi-upright |
| Productivity | high | high | high |
| Self fruitfulness | yes | yes | yes |
| Number of young | medium | medium | medium |
| shoots | | | |
| CANES | | | |
| Primocanes | | | |
| Anthocyanin | present | present | present |
| coloration | | | |
| Spines | present | present | absent |
| color | green | purple | — |
| attitude of tip | downward | horizontal | — |
| texture | rigid | heavy | — |
| presence and | present; | present; | absent |
| distribution on | irregularly | irregularly | |
| petioles | distributed | distributed | |
| density in central | medium | medium | — |
| third of shoot | | | |
| Internodal distance | 2.5 | 2.6 | 3.1 |
| (cm) - central third | | | |
| of mature cane | | | |
| Glaucosity on full | absent to very | weak | weak |
| grown shoot | weak | | |
| Strength of full | medium | medium | strong |
| grown shoot | | | |
| Cane cross section | angular to | rounded to | angular to |
| | grooved | angular | grooved |
| LEAVES | | | |
| Relief between veins | weak | medium | medium |
| Number of leaflets | usually 5 | usually 3 | usually 5 |
| Leaf color | medium | medium | light |
| upper side | 137A, 137B | 137A, 137B | 147A |
| underside | 137C, 138A | 147B | 146A |
| Glossiness of upper | medium | medium | dull |
| surface | | | |
| Leaf cross section | concave | concave-flat | concave |
| Terminal leaflet | | | |
| length (cm) | 10.3 | 8.9 | 11.1 |
| width (cm) | 7.6 | 7.6 | 9 |
| shape | ovate | cordate | cordate |
| tip | acute | acuminate | acuminate |
| base | acute | cordate | cordate |
| margin | double serrate | double serrate | double serrate |
| Lateral leaflet | | | |
| overlap of lateral | free to | overlapping | overlapping |
| leaflets | touching | | |
| length (cm) | 9.9 | 8.7 | 10.2 |
| width (cm) | 6.3 | 6.1 | 7.1 |
| shape | ovate | ovate | ovate |
| tip | acuminate | acuminate | acute |
| base | acute | acute | acute |
| margin | double serrate | double serrate | serrate |
| Petiole | | | |
| mean length (cm) | 8.4 | 5.3 | 7.9 |
| range | 5.2-10.6 | 3.6-8.7 | 3.9-10.2 |
| pigmentation of | reddish | green - slightly | purple |
| upper surface | | pink | |
| pigmentation of | green | green - slightly | green - |
| underside | | pink | pinkish |
| Length of stalklet | short | very short | medium |
| Rachis length (cm, | 3.6 | 2.8 | 3.1 |
| between terminal and | | | |
| adjacent lateral | | | |
| leaflets) | | | |
| Stipule orientation | erect | variable; | erect |
| | | clasping to | |
| | | erect | |
| FLOWERS | | | |
| Time of bud burst | early | early | late |
| Time of beginning of | early | early | late |
| flowering | | | |
| Flower size | medium to | small to | small to |
| | large | medium | medium |
| Petal size | | | |
| length (mm) | 19.1 | 16.5 | 18.3 |
| width (mm) | 12.4 | 11.7 | 10.9 |
| Anthocyanin color | absent | absent | present |
| of pedicel | | | |
| Intensity of | — | — | weak |
| pedicel coloration | | | |
| Length of pedicel | medium to | long | short |
| | long | | |
| Flower number (third | 5.8 | 3.6 | 2 |
| node from tip of | | | |
| lateral) | | | |
| FRUIT | | | |
| Harvest season | early | early | mid to late |
| Dimensions | 4.2 | 5.2 | 3.2 |
| weight (g/fruit) | | | |
| size | medium | medium | small |
| length (cm) | 2.7 | 3.3 | 1.9 |
| width (cm) | 2.1 | 1.4 | 1.9 |
| Fruiting lateral length | medium | medium | medium - long |
| (in mid cane) | | | |
| Mean number of fruit | 7.7 | 6.2 | 22.8 |
| per lateral | | | |
| range | 5-20 | 3-9 | 17-40 |
| Shape | ovate to | narrow ovate; | round to |
| | elliptic; | much longer | ovate; |
| | longer than | than broad | as long as |
| | broad | | broad |
| Color | black | purple-black | black |
| | | to black | |
| immature | 184A | 178A-183B | 184A |
| maturing | 187A | 187A | 200A-202A |
| mature | 200A | 200A | 202A |
| Firmness | firm | medium | firm |
| Glossiness | strong | medium - | medium |
| | | strong | |
| Soluble solids | 9.6 | 9.7 | 9.9 |
| Titratable acidity | 9 | 13.3 | 9.9 |
| (% as citric acid) | | | |
| (ml of added .1 N | | | |
| NaOH to pH 8.1) | | | |
| Number of drupelets | 85 | 86.12 | 40 |
| per fruit | | | |

### 5.2 NUCLEIC ACID FINGERPRINTING

Distinctive patterns of polymorphism can be detected using a variety of nucleic acid analysis methods. In one non-limiting example, molecular genetic maps can be produced using random amplified polymorphic DNA (RAPD) (Williams et al., 1990, “DNA polymorphisms amplified by arbitrary primers are useful as genetic markers”, Nucleic Acids Res. 18(22):6531-5). Using a variety of oligonucleotide primers, alone or in combination, RAPD analysis of Zorro, Chester, and Olallie yielded DNA fragment patterns that uniquely distinguish each of these genetically distinct genotypes.

We claim:

1. A new and distinctive cultivar of blackberry plant, substantially as shown and described.
